(19) United States
(12) Patent Application Publication
(10) Pub. No.: US 2015/0000675 A1
(43) Pub. Date: Jan. 1, 2015

(54) NASAL BREATHING AID

(71) Applicants: JAMES KALLIKOUNIS, LAGUNA BEACH, CA (US); ANDREW KALLIKOUNIS, LAGUNA BEACH, CA (US)

(72) Inventors: JAMES KALLIKOUNIS, LAGUNA BEACH, CA (US); ANDREW KALLIKOUNIS, LAGUNA BEACH, CA (US)

(21) Appl. No.: 13/930,551

(22) Filed: Jun. 28, 2013

(51) Int. Cl. A61F 5/08; A61F 5/56

(52) U.S. Cl. CPC A61F 5/08; A61F 5/56
USPC 128/848; 606/199

(57) ABSTRACT

A nasal breathing aid includes a base portion and two extending diverging arms. The arms, near their base include inward facing contact elements which are adapted to “pinch” the vomer bone within the nose. The arms, at their outer ends, include outward facing contact elements which are adapted to apply pressure to the inner wall of the nostrils, urging a wider passageway through the nostrils.

### BACKGROUND OF THE INVENTION

1. Field of the Invention

The present invention relates to devices to enhance respiration and, more particularly to a device that works in the human nose to facilitate breathing and to alleviate snoring.

2. General Background and State of the Art

Prior art devices to improve breathing and suppress snoring have included adhesive strips which are applied to the nose and which include a spring like device that tends to open the nostrils. Another approach entails a mouthpiece device which holds the mouth slightly open. While these devices have achieved a degree of popularity, their efficacy for suppressing snoring and enhancing breathing has been questioned. Yet another device, disclosed in U.S. Pat. No. 5,713,833 to Milligan, applies a controlled pressure to the nerves of the septum which pressure is intended to stimulate the nerves to provide relief to secretion and breathing problems. Apparently, the device has not achieved the success envisioned by the inventor.

What is needed and what is provided by the present invention is a simple device that assists breathing and ameliorates the snoring problem with a simple device that works within the human nose.

### INVENTION SUMMARY

According to the present invention, a device is provided which can be worn in the nose and which is made of a soft, resilient material that will not chafe or irritate the nasal passages. A base element is designed to rest between the upper lip and the maxilla of the septum so that a slight pressure is exerted upon the vomer. A pair of arms extends from the base portion. The arms are biased into contact with the vomer near the base but flare outwardly and are biased into contact with the interior surface of the upper portion of the nostril nasal passage.

As a result, there is a simultaneous upward pressure and a “pinching” of the vomer while the upper nostrils are urged outwardly to maintain open nasal passages. As one knows, a sneeze can sometimes be suppressed by placing the finger under the base of the nose and pressing upward. This places a pressure upon the vomer which tends to suppress a sneeze. It has been found that the combination of pressures tends to prevent snoring and enhances the airflow through the nasal passages.

In alternative embodiments, the structure of the device of the present invention can have hemispheric protrusions at the points of contact or other shapes to exert the slight pressures on the nasal structures for optimum effect. It is believed that the desired effect is achieved through the simultaneous application of upward pressure on the septum, a “pinching” pressure on the vomer and an outward pressure on the inner surface of the nostrils. It is believed that the serendipitous combination of the “Breathe Right®” nasal strip, the Hannigan Septum Stimulator and means for applying an upward pressure on the vomer through the maxilla that achieves the goals of enhanced breathing and snore suppression.

The novel features which are characteristic of the invention, both as to structure and method of operation thereof, together with further objects and advantages thereof, will be understood from the following description, considered in connection with the accompanying drawings, in which the preferred embodiment of the invention is illustrated by way of example. It is to be expressly understood, however, that the drawings are for the purpose of illustration and description only, and they are not intended as a definition of the limits of the invention.

### BRIEF DESCRIPTION OF THE DRAWINGS

FIG. 1 is a plan view of a breathing aid according to the present invention;

FIG. 2 is a side sectional view of the breathing aid of FIG. 1;

FIG. 3 is plan view of an alternative embodiment of the breathing aid of the present invention;

FIG. 4 is a frontal view of the human nose; and

FIG. 5 is a sectional view of the nose of FIG. 4 taken along line 5-5 in the direction of the appended arrows.

### DETAILED DESCRIPTION OF THE PREFERRED EMBODIMENTS

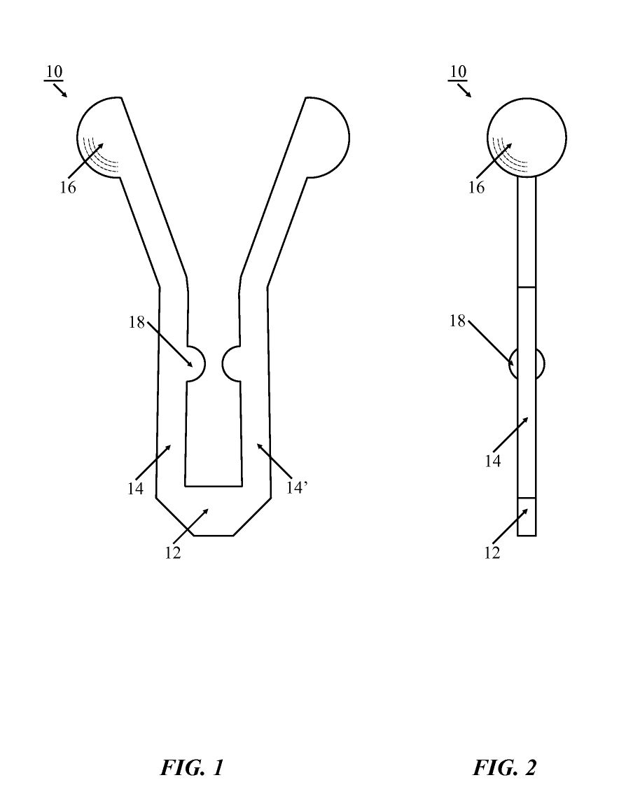

Turning first to FIGS. 1 and 2, there is shown a breathing aid 10 according to a preferred embodiment of the present invention. The breathing aid 10 includes a base portion 12 and arms 14, 14′ extending outward therefrom. As shown, the arms 14, 14′ flare outwardly and terminate in an outward facing rounded protuberance 16 In the preferred embodiment, the protuberances 16 can be hemispherical.

On the inner surface of the arms 14, 14′ is a second pair of inward facing protuberances 18 adjacent the base portion 12. As with the outward facing protuberances 18, in the preferred embodiment, the inward facing protuberances are also hemispherical.

The arms 14, 14′ are biased inwardly in the region of the inward facing protuberances 18 so that they are configured to grip the cartilage adjacent the vomer or, preferably, the vomer, itself. The first pair of outward facing protuberances 16 are outwardly biased to apply a slight pressure to the inner walls of the nostril, urging them outward.

It is contemplated that the breathing aid of the present invention will be made of a silicon rubber of a density sufficient to provide the bias necessary to engage the vomer and open the upper nasal passages. In the preferred embodiment, depending upon the age of the intended user and the dimensions of the nose, the spacing of the arms 14, 14′ at the base is approximately ¼″. The spacing between the arms 14, 14′ at the outer ends is approximately 1″. The approximate length of the arms 14, 14′ is ⅞″.

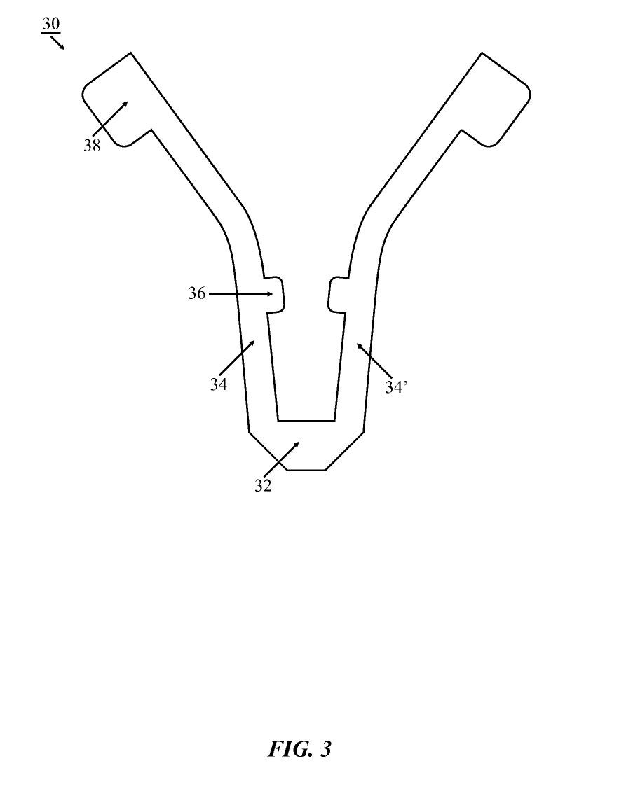

Turning next to FIG. 3, there is shown an alternative embodiment of the breathing aid 30 of the present invention. As with the device of FIGS. 1 and 2, it includes a base portion 32 and a pair of diverging arms 34, 34′. Adjacent the base portion are inward facing contact elements 36 and, at the outer ends a pair of outward facing contact elements 38. In this embodiment, the contact elements 36, 38 are rounded but not necessarily hemispherical.

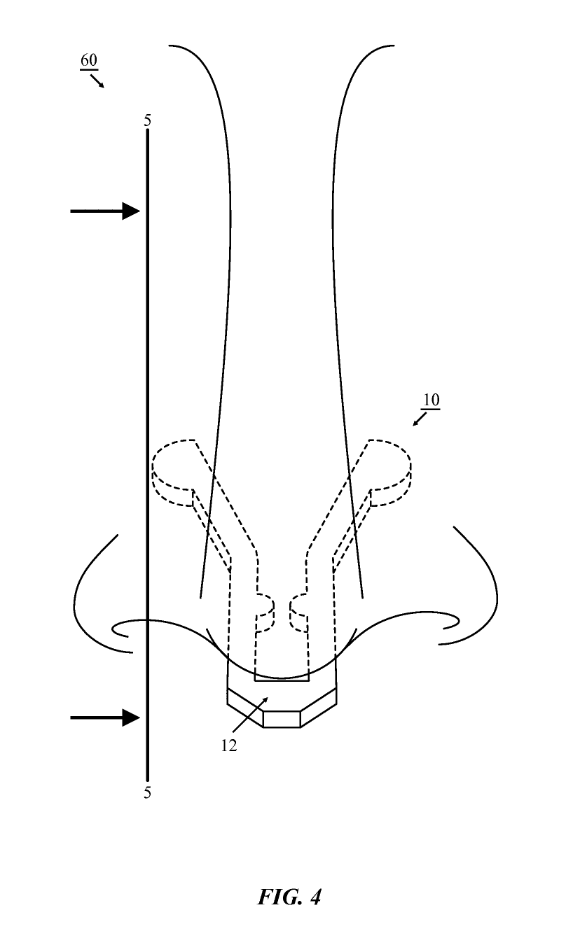

FIG. 4 is a frontal view of a human nose 60 with the device 10 of the present invention inserted. As can be seen, the base portion 12 extends below the base of the nose.

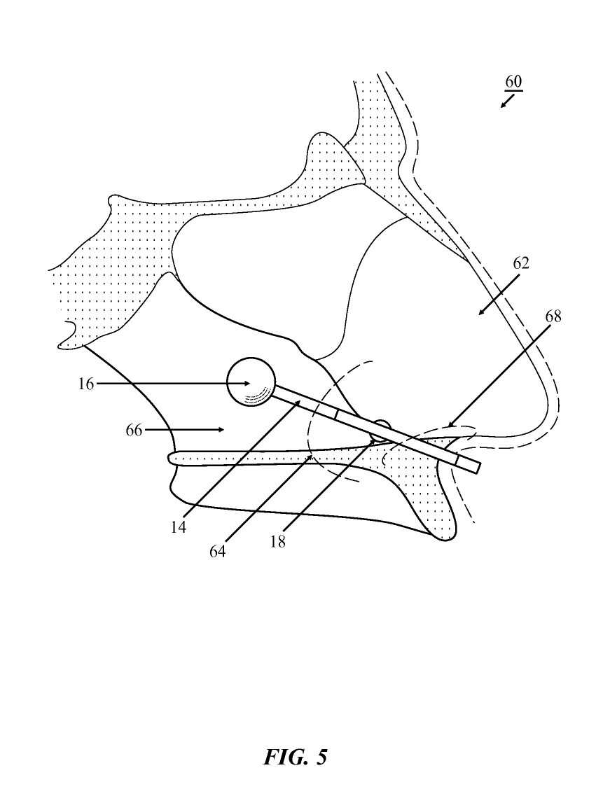

Turning finally to FIG. 5, there is shown a side view of the human nose 60. A central cartilage 62 divides the nose into left and right nostrils 64. The vomer 66 is a flat bone behind the cartilage 62 at the base of the nose 60. Within the nostrils 64 are nasal passages 68, through which normal breathing takes place. However, through minor inflammation, the upper portions of the nasal passages 68 become constricted. It has been found that the outer protuberances 16 of the arms 14 of the breathing aid can widen the nasal passages while the inner portion protuberances 18 of the arms 14 apply slight pressure to the vomer 66 which tends to suppress snoring. Further, the widening of the nasal passages encourages a greater airflow through the nose, reducing the need to breathe through the mouth, which may exacerbate the snoring response.

While the specification describes particular embodiments of the present invention, those of ordinary skill can devise variations of the present invention without departing from the inventive concept.

We claim:

1. A nasal breathing aid comprising:
a. a base portion;
b. a pair of diverging arms extending outwardly;
c. a pair of outward facing protuberances at the terminal ends of said arms; and
d. a pair of inward facing protuberances at the inner ends of said arms adjacent said base portion,
whereby, said breathing aid, in use, has said base portion resting on a wearer's upper lip with the inward facing protuberances engaging the vomer of the wearer and the outward facing protuberances engaging the inner surfaces of the wearer's nostrils.

2. The nasal breathing aid of claim 1 wherein said protuberances are hemispherical.

3. The nasal breathing aid of claim 1 wherein said protuberances are rounded inward extensions.

4. The nasal breathing aid of claim 1 wherein the outer ends of said arms are biased outwardly for engagement of said outward protuberances with the inner surfaces of a wearer's nostrils.

5. The nasal breathing aid of claim 1 wherein the inward facing protuberances are biased inwardly for engagement with the vomer of the wearer's nose.

6. A nasal breathing aid comprising:
a. a base portion;
b. a pair of arms having a first portion and a second portion, said first portion extending substantially orthogonal to said base portion and having an inward bias and said second portion diverging outwardly and having an outward bias;
c. a pair of inward facing protuberances at the inner ends of said first portion of said arms adjacent said base portion; and
d. a pair of outward facing protuberances at the outer ends of said second portion of said arms adjacent the outer ends thereof.
whereby, said breathing aid, in use, has said base portion resting on a wearer's upper lip with said inward facing protuberances biased into engagement with the vomer of the wearer and said outward facing protuberances biased into engagement with the inner surfaces of the wearer's nostrils.

7. The nasal breathing aid of claim 6 wherein said protuberances are hemispherical.

8. The nasal breathing aid of claim 6 wherein said protuberances are rounded extensions.

9. A method of ameliorating snoring and enhancing breathing in a subject comprising the steps of simultaneously pinching the vomer and exerting a slight upward pressure upon the vomer of the subject and exerting an outward force on the upper areas of the subject's nasal passages.
